(19) United States
(12) Patent Application Publication
(10) Pub. No.: US 2020/0163599 A1
(43) Pub. Date: May 28, 2020

(54) CLOUD BASED PULSE OXIMETRY SYSTEM AND METHOD INCLUDING OPTIONAL HEADBAND COMPONENT

(71) Applicant: Serenium, Inc., Palo Alto, CA (US)

(72) Inventors: David Gozal, Chicago, IL (US); David Rosen, Glencoe, IL (US); Michael Cory Zwerling, Palo Alto, CA (US)

(73) Assignee: Serenium, Inc., Palo Alto, CA (US)

(21) Appl. No.: 15/779,383

(22) PCT Filed: Sep. 19, 2016
(86) PCT No.: PCT/US2016/052479

§ 371 Date: May 25, 2018

Related U.S. Application Data
(60) Provisional application No. 62/219,877, filed on Sep. 17, 2015.

(51) Int. Cl. A61B 5/1455; A61B 5/00; G16H 40/67; G16H 40/63

(52) U.S. Cl. CPC A61B 5/14551; A61B 5/6814; G16H 40/63; G16H 40/67; A61B 5/0022; A61B 5/4818

(57) ABSTRACT

A cloud based pulse oximetry system and method is provided that incorporates both a software and hardware component. The hardware component includes a limited use pulse oximetry probe having an energy source and data transmitting capabilities. The probe houses a light source and a light detector that compare and calculate the differences in the oxygen-rich versus oxygen-poor hemoglobin in the body part to which, it is attached. Data from the probe is collected, analyzed, and communicated using a cloud based computing system. The system may be used for collecting and analyzing pulse oximetry data for any medical need such as diagnosis of obstructive sleep apnea.

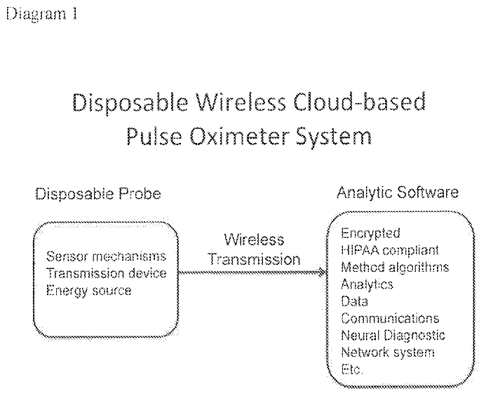

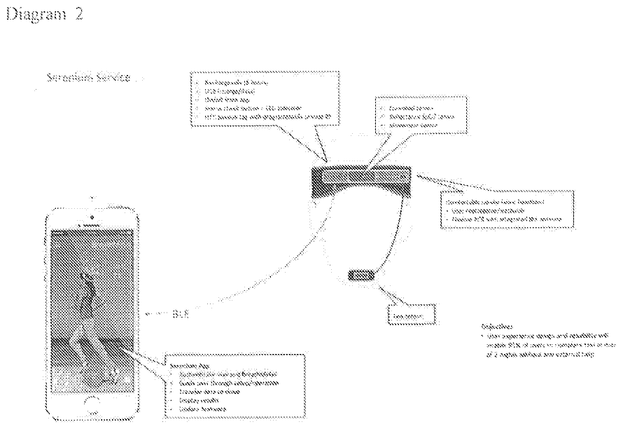

### FIELD OF THE INVENTION

This invention relates to methods, systems, and apparatus for using pulse oximetry to measure blood oxygen saturation. Specifically, it relates to such methods, systems, and apparatus using disposable wireless cloud based pulse oximetry systems to identify and diagnose potential obstructive sleep apnea in a patient.

### BACKGROUND OF THE INVENTION

Obstructive sleep apnea-hypopnea syndrome (OSAHS) is characterized by repetitive occlusion of the upper airway during sleep, causing intermittent cessations of breathing (apneas) or reduction in airflow (hypopneas). Events of apnea are accompanied by hypoxemia and bradycardia. They are often terminated in arousals, and the resulting sleep fragmentation can lead to excessive daytime sleepiness. As a result, OSAHS has been pointed out as a major public health concern. Additionally, long-term effects are related to the cardiovascular system, including hypertension, arrhythmias, congestive heart failure and cerebrovascular disease.

The “gold standard” approach to diagnose OSAHS is overnight polysomnography (PSG). However, PSG has several limitations since it is both complex and costly due to the high number of physiological signals that need to be recorded. It must be performed in a special sleep unit and under supervision of a trained technician. PSG monitors different physiological recordings such as electrocardiogram (ECG), electroencephalogram (EEG), electrooculogram (EOG), electromyogram (EMG), oxygen saturation, abdominal ventilatory effort and snoring. These recordings must be subsequently analyzed by a medical expert to obtain a final diagnosis. Despite its high diagnostic performance, PSG presents some drawbacks since it is complex, expensive and time-consuming. Additionally, all the PSG signals need offline inspection in order to derive the apnea-hypopnea index (AHI), which is used to establish whether OSAHS is present and its severity. As a result, research recently has focused on the development of alternative and simpler diagnostic techniques, such us the use of medical systems based on nocturnal pulse oximetry. An interesting approach is the analysis of single-channel sleep-related recordings, which reduces cost and complexity. In this regard, automated processing of oximetry signals is a promising alternative due to its reliability, simplicity, and suitability.

Nocturnal pulse oximetry allows to monitor respiratory dynamics during sleep by measuring blood oxygen saturation (SpO2). This recording provides useful information about OSAHS. Events of apnea are characterized by a decrease in the SpO2 value, which reflects airflow reduction and hypoxemia. Subsequently, respiration is restored and the saturation value increases until its baseline level. As a result, SpO2 signals from OSAHS patients tend to be more unstable than those from control subjects due to the recurrence of apneas during sleep. This different behavior can be exploited to diagnose OSAHS.

Although many advances have been made utilizing pulse oximetry to diagnose OSAHS, known and currently utilized probes can be cumbersome, expensive and not as accessible to patients for usage. There remains a need for a pulse oximetry system that is more approachable, less expensive, and available for usage in a variety of situations. The system of the present invention, accordingly, contemplates a lower cost system that uses a disposable oximetry probe in conjunction with a cloud based computing system to measure, analyze, and communicate data and results in a more efficient and economical manner.

### SUMMARY OF THE INVENTION

While the invention may be susceptible to embodiment in different forms, there is shown in the drawings, and herein will be described in detail, specific embodiments with the understanding that the present disclosure is to be considered an exemplification of the principles of the invention, and is not intended to limit the invention to that as illustrated and described herein.

While preferred embodiments of the present invention are shown and described, it is envisioned that those skilled in the art may devise various modifications of the present invention without departing from the spirit and scope of the appended claims.

With reference to Diagram 1, the system of the present invention contemplates the capturing of vital sensor data and analyzing data utilizing a limited use and/or disposable wireless probe and a cloud based computing system to capture, analyze, and transmit/communicate the data and analytical results. The system contains hardware and software components as will be described herein.

A. Hardware

Pulse oximetry is a procedure used to measure the oxygen level (or oxygen saturation) in the blood. It is considered to be a noninvasive, painless, general indicator of oxygen delivery to the peripheral tissues (such as the finger, earlobe, or nose). A clip-like device called a probe is placed on a body part to measure the blood that is still carrying or is saturated with oxygen. The probe typically houses a light source, a light detector, and may include a microprocessor, which compares and calculates the differences in the oxygen-rich versus oxygen-poor hemoglobin. A typical probe has one side having a light source with two different types of light, infrared and red, which are transmitted through the body part, such as a finger, to the light detector side of the probe. The oxygen-rich hemoglobin absorbs more of the infrared light and the hemoglobin without oxygen absorbs more of the red light.

For the present invention, a pulse oximeter probe is utilized that is placed on the fingertip (in multiple sizes, small, medium, large adult, and pediatric/infant sizes) or another part of the human body (such as ear, toe or forehead) which captures blood oxygen levels and heart rate or other vital signs. Preferably, the probe is designed for limited use and is a disposable device having its own energy source and sensor mechanism(s) (as are known in the art) to measure blood oxygen levels. The probe contemplated for use with the present invention also preferably does not include a microprocessor but rather relies on a cloud computing system to store and analyze the data.

Also, the probe utilized with the present invention preferably emits a wireless transmission signal of the data being monitored using Bluetooth, radio frequency or other suitable and known wireless transmission method. These data readings are then preferably captured by a mobile device such as a phone or tablet or the like and uses the supporting application or software residing on such mobile device to analyze and interpret the data. This data is then transmitted and uploaded into a cloud computing system for data storage and analysis. Such data are preferably securely accepted with a unique identifier or “hand-shake” procedure.

Depending on the energy source or battery life, the disposable device can be contemplated for single usage or for multiple uses based on a limited energy or battery source in the unit, and then disposed or recycled.

Upon initial use of the probe, the probe preferably emits a test transmission signal using Bluetooth, radio frequency or other wireless transmission to insure authentication and secure connection with the cloud-based system. The supporting application or software for the mobile device will preferably confirm this initialization process or pre-test operations and if needed identify trouble shooting procedures.

A.1 Optional Headband Component

The probe optionally may at least in part comprise a headband component and related system. The headband system, as shown in Diagram 2, may also generally include a system for capturing vital sensor data from the wearer and may analyze the data using a magnet with a wireless sensor positioned on the forehead and chin to deliver clinically relevant information.

Known prior art devices, from which the present invention builds from, include a distance measuring device comprising an emitter and a receiver. The prior art emitter is preferably arranged to produce a magnetic field with a resonant circuit having a resonant frequency. The prior art receiver is preferably arranged to pick up at the resonant frequency the magnetic field emitted by the emitter and convert the strength of the magnetic field picked up into a first signal having an energy value. The prior art emitter is preferably arranged to produce the magnetic field intermittently with each emission having a predetermined energy. The prior art receiver is preferably connected to a detector arranged to determine a distance measurement signal representing the distance between the emitter and the receiver.

According to these prior art devices, the strength of the magnetic field picked up gives a measurement of the distance between the emitter and the receiver and can in this way be used to measure a distance between two points. To obtain this distance the first signal is amplified selectively. One drawback of the known devices is that they are not adapted to reliably and accurately measure distances of more than a few centimeters without having to use a high-strength magnetic field. Moreover the selective amplification of the first signal is not stated precisely, such that it does not allow an accurate determination of the distance, in particular when the first signal comprises an amount of noise and interference. For this reason earlier known devices have been found to be unreliable to measure movements of the mouth of a living being in applications where high resolution is required. This is because high-power magnetic fields are not suitable to be used frequently on living beings without having a detrimental effect on the health of that living being.

U.S. Pat. No. 8,203,330 discloses a distance measuring device that is capable of measuring distances very accurately, in particular on the human body, without having to use a value of magnetic field that would be too strong for the human body that addresses some of the shortcomings of the prior art systems. The '330 patent is issued to Nomics and the present applicant Serenium Inc. has obtained an exclusive license to practice the invention in the United States together with any Serenium improvements. As such, the entirety of U.S. Pat. No. 8,203,330 is incorporated herein by reference.

Specifically, the '330 device is characterized in that the detector is arranged to determine the distance measurement signal by correlation of the first signal with a second predetermined signal having a waveform representative of a signal to be picked up by the receiver. The second signal preferably comprises a time window having a predetermined duration and comprising at least an initial sub-period, an intermediate sub-period and a final sub-period. The second signal preferably being an alternating signal synchronized with the first signal and whereof the amplitude is attenuated during the initial and final periods and substantially at a maximum during the intermediate period. The use of an alternating signal whereof the amplitude is attenuated during the initial and final sub-periods makes it possible to considerably reduce the noise and interference appearing in frequency ranges far from the resonant frequency. The fact that the amplitude is substantially at a maximum during the intermediate period, that is to say where the first signal reaches its maximum value, makes it possible to considerably reduce the noise and interference in the frequency ranges very close to the resonant frequency whilst making maximum use of the amplitude of this signal during this intermediate sub-period and therefore to be able to work with magnetic fields whereof the power remains low and therefore without damage to the human body. The '330 device finds its application in detectors for sleep disorders or other forms of illness.

A first preferential embodiment of the '330 device is characterized in that the detector is arranged to implement the correlation by multiplication and integration with the second signal, which second signal is formed by said waveform representing a sinusoidal waveform in synchronization with the first signal itself multiplied by a Tukey window with reduced taper factor. This allows rejection of noise and interference outside the detection frequency.

A second preferential embodiment of the '330 device according to the invention is characterized in that the emitter is housed in a case and arranged to produce said magnetic field outside said case with a power less than 1 mTesla, preferably less than 1 μTesla. This embodiment is particularly adapted to the human body. A sleep disorder detector according to the '330 system is characterized in that the device is mounted on a support arranged to be applied onto the head of a living being so as to measure movements of the mouth.

The headband system of the present invention builds on the prior art systems described above in this section and addresses some of the shortcomings thereof. Specifically, and with reference to Diagram 2, the present headband system comprises many of the hardware and software components of the '330 patent, but has been improved and advantageously reconfigured for consumer or user benefits and as so as to fit into an embodiment of a headband system configuration as shown in Diagram 2. In general, the system preferably has at least two variations: a basic headband system and a system with an optional “built in” pulse oximeter probe.

In the present invention, the main distance measuring unit is integrated or attached to a headband to be worn over a person's forehead. There is an attaching magnet unit that sits on the chin and can be either hard wired or wirelessly connected (via Bluetooth, radio frequency, or other wireless transmission configurations) with the main forehead unit.

The main unit contains comprises an emitter and a receiver placed at a distance of the headband and the chin magnet. The emitter comprises an induction coil preferably connected in series with a capacitor, whilst in the receiver the induction coil and the capacitor are preferably connected in parallel. The emitter and receiver may be connected by means of a cable to a conditioning and measuring unit. The unit comprises a detector and an energizing circuit. The sensors use the property that a resonant circuit has of energizing another one tuned to the same frequency, through their mutual induction coil. The use of resonant circuits rather than simple induction coils significantly improves both the performance of the energizing circuit and the sensitivity of the sensor. The use of simultaneous connection of the emitter and the receiver to the same electronic circuit makes it possible to simplify the device by avoiding synchronization errors.

The main unit also may comprise a pulse oximeter sensor that attaches around the user's forehead through the headband, appropriate pulse oximeter system boards, and associated components and devices.

The main unit preferably comprises Bluetooth or radio frequency or other wireless transmission capabilities for wireless connections captured by a mobile device like a phone or other similar device and uses the supporting App or Software residing on such mobile device. Similar to the other oximeter probe configurations described above, the data from the pulse oximeter is preferably transmitted into a cloud computing system for data storage and analysis, where the data are securely accepted with a unique identifier or hand-shake procedure.

There may be Bluetooth connectivity between the main forehead unit and the magnet on the chin or, as described above, a hard wire connection between these two components. There may also be a USB plug for hard wire connectivity and potential recharging.

This system could be a disposable unit with a single usage or for multiple uses based on a limited energy or battery source in the unit, and then disposed or recycled. Alternatively, it could also be rechargeable.

Upon initial use of the headband system, the main unit preferably emits a test transmission signal using Bluetooth, radio frequency or other wireless transmission to insure authentication and secure connection with the cloud-based system. The supporting App or Software for the mobile device will confirm this initialization process or pre-test operations and if needed identify trouble shooting procedures.

B. Analytics Software

Software configured or known in the art is used with the data that is collected and transmitted from the probe to the cloud based system (or server based system) for analysis and is more preferably encrypted and HIPAA compliant. It should be appreciated that the software preferably utilizes and includes one or more of method algorithms known in the art, data analytical tools, communications capabilities to communicate the analysis and/or raw data results, and neural diagnostic network systems.

What is claimed is:

1. A wireless cloud computing based pulse oximeter system and method as shown and described, in any configuration and in any combination.
